(19) United States
(12) Plant Patent Application Publication
(10) Pub. No.: US 2005/0216982 P1
(43) Pub. Date: Sep. 29, 2005

(54) PECAN TREE 'EXCEL' VARIETY

(50) CARYA ILLINOINENSIS; PECAN

(76) Inventor: Andy Clough, Blackshear, GA (US)

Correspondence Address: STOCKWELL & ASSOCIATES, PSC, 861 CORPORATE DRIVE, SUITE 201, LEXINGTON, KY, 40503, US

(21) Appl. No.: 10/806,673

(22) Filed: Mar. 23, 2004

(51) Int. Cl. A01H 5/00

(52) U.S. Cl. PLT/153

(57) ABSTRACT

The new variety “EXCEL” pecan tree is an early bearing tree with fruit maturing and ready for harvest about the 1^{st} week in October. The new “EXCEL” tree actually blooms out later than the popular “Stuart” variety yet this new variety actually produces mature nuts that ripen earlier. The tree displays a semi-dwarf type growth with small twiglets, yet the nut has the general size and shape of the “Desirable” pecan variety. The fruit has a thick shell and an excellent quality that yields about 51%. The final bloom is red and the catkins are long and thin. The tree has also shown to be disease resistant as well as insect resistant for the foliage. The foliage is somewhat sparse, which allows for better sunlight penetration and air circulation. On the worst of conditions, the “EXCEL” has shown no scab on the nuts. The tree is precocious (early bearing) as well as prolyfic (heavy bearing).

### FIELD OF THE INVENTION

This invention relates generally to a new and distinct variety of pecan tree.

### BACKGROUND OF THE INVENTION

The present invention relates to a new and distinct variety of pecan tree discovered growing by the inventor on his farm in Pierce County, Ga. The instant tree is selected from seedlings of unknown parentage and will be known commercially as the “EXCEL” variety and known scientifically as Carya illinoinensis.

### BRIEF SUMMARY OF THE INVENTION

The tree of the new “EXCEL” variety was found growing in a planted orchard on the inventor's farm in the year of 1990. The tree was estimated to be between about 20 and 25 years old at the time of discovery. The inventor's attention was attracted to the original tree of the new “EXCEL” variety because of its superior characteristics.

In particular, the new variety “EXCEL” pecan tree is an early bearing tree with fruit maturing and ready for harvest about the 1^{st} week in October, which puts this new variety at about 2 weeks earlier that the standard “Stuart” variety. Surprisingly, the new “EXCEL” tree actually blooms out later than the popular “Stuart” variety yet this new variety actually produces mature nuts that ripen earlier.

The new “EXCEL” tree displays a semi-dwarf type growth with small twiglets, yet the nut has the general size and shape of the “Desirable” pecan variety. The fruit has a thick shell and an excellent quality that yields about 51%. The final bloom is red and the catkins are long and thin. At this time the new variety is thought to be a self-pollinator.

The “EXCEL” tree has also shown to be disease resistant as well as insect resistant for the foliage. The foliage of this new variety is somewhat sparse, which allows for better sunlight penetration and air circulation. On the worst of conditions, the “EXCEL” has shown no scab on the nuts. The tree is precocious (early bearing) as well as prolyfic (heavy bearing).

Trees of the new “EXCEL” variety have been asexually reproduced by grafting and budding by the inventor at his farm in Pierce County Ga. Asexual reproduction has shown that the foregoing characteristics and distinctions survive and are established and transmitted through successive propagations as witnessed by University of Georgia Plant Pathologist, Dr. Patrick Conner, of Athens, Ga.

### BRIEF DESCRIPTION OF THE PHOTOGRAPHS

The photographs show various aspects of the new “EXCEL” variety while being grown at Pierce County near Blackshear, Ga., wherein the color is illustrated as true as is reasonably possible in a depiction of this character.

FIG. 1 shows the generally upright and spreading sturdy growth habit of the original tree of the new “EXCEL” variety when observed during 2001.

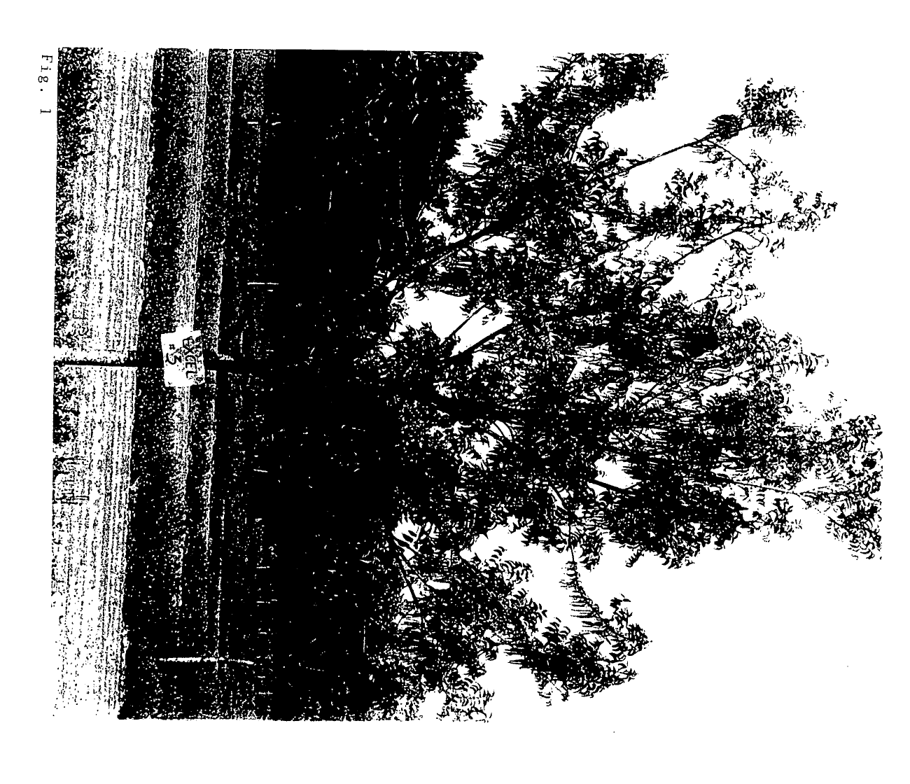

FIG. 2 shows typical foliage and nuts of the new “EXCEL” variety during spring, summer and fall of 2002.

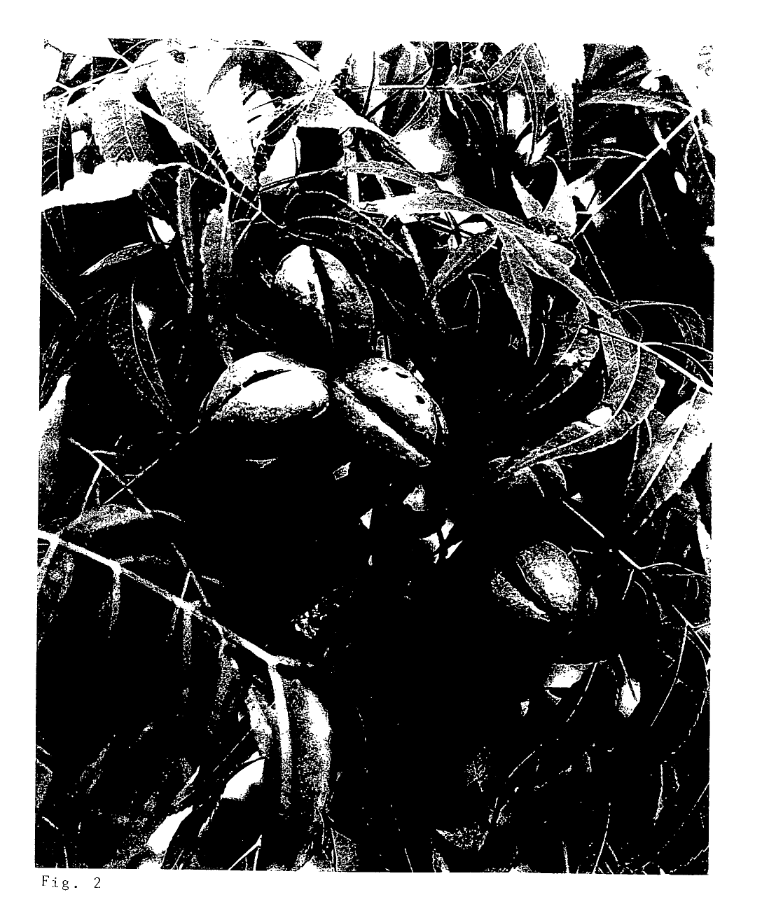

FIG. 3 illustrates typical nuts in the shell of the new “EXCEL” variety following removal from the husks.

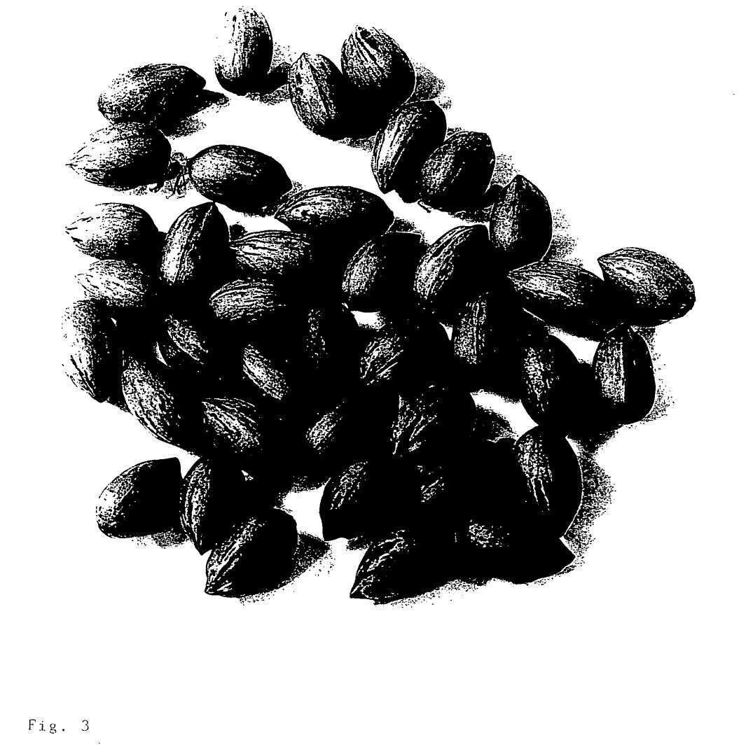

FIG. 4 illustrates typical nuts of the new “Excel” variety showing the kernels following removal from the shell.

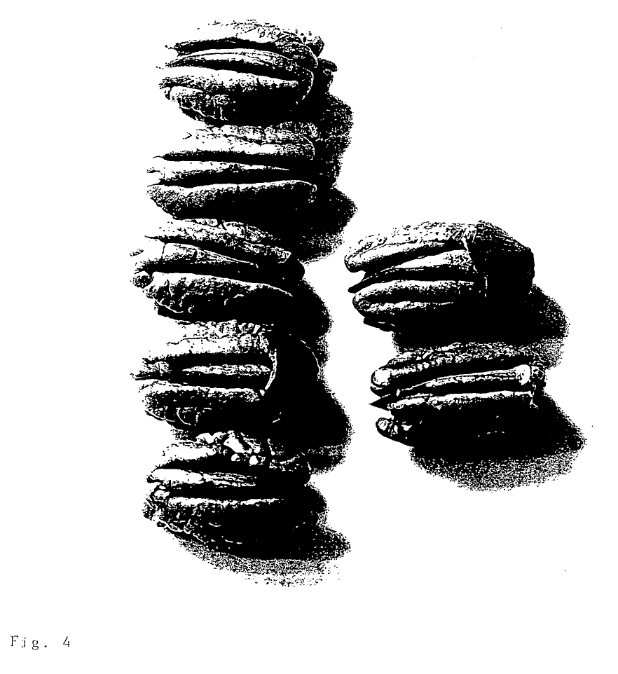

FIG. 5 illustrates typical foliage, buds and catkins of the new “EXCEL” variety when the tree is in bloom.

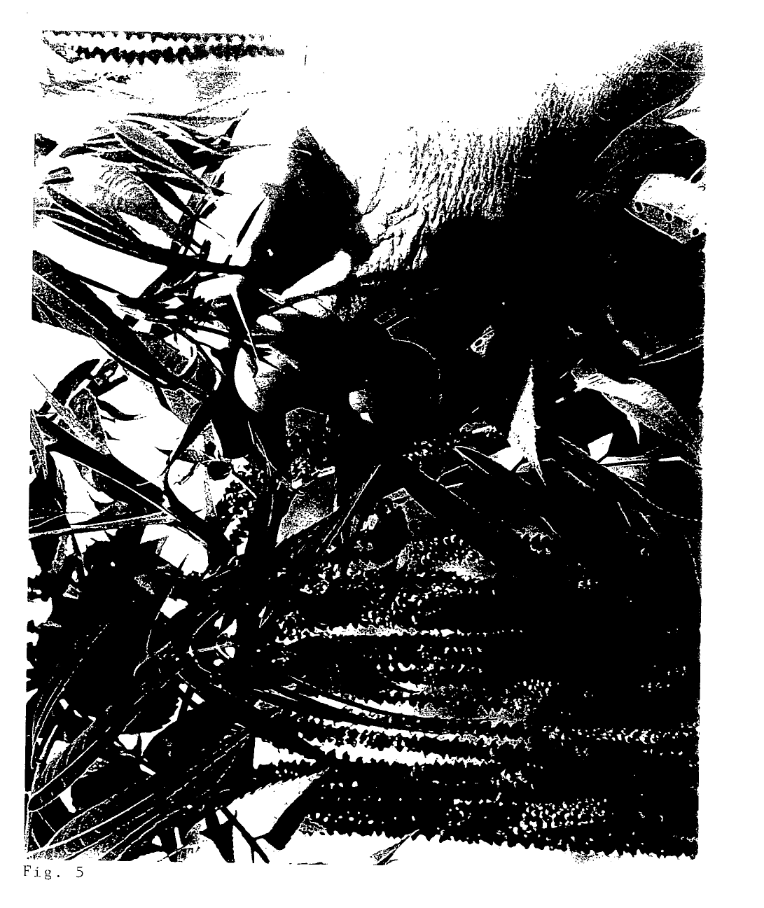

FIG. 6 is a close up picture illustrating the typical bloom of the new “EXCEL” variety.

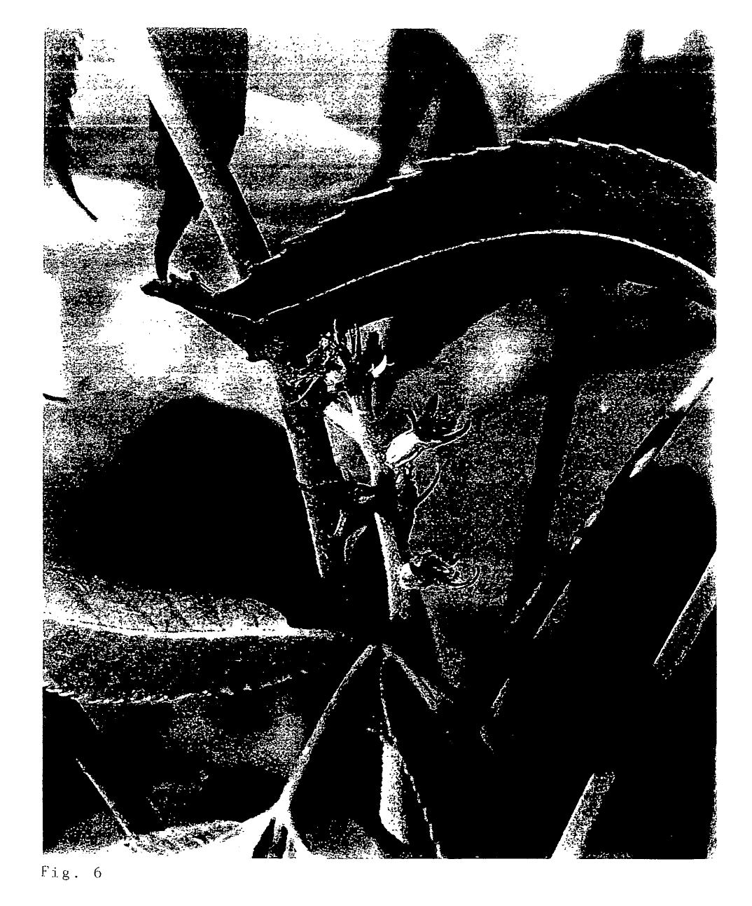

FIG. 7 illustrates the open tube growth of the branches of the new “EXCEL” variety taken during the winter, while the tree is dormant.

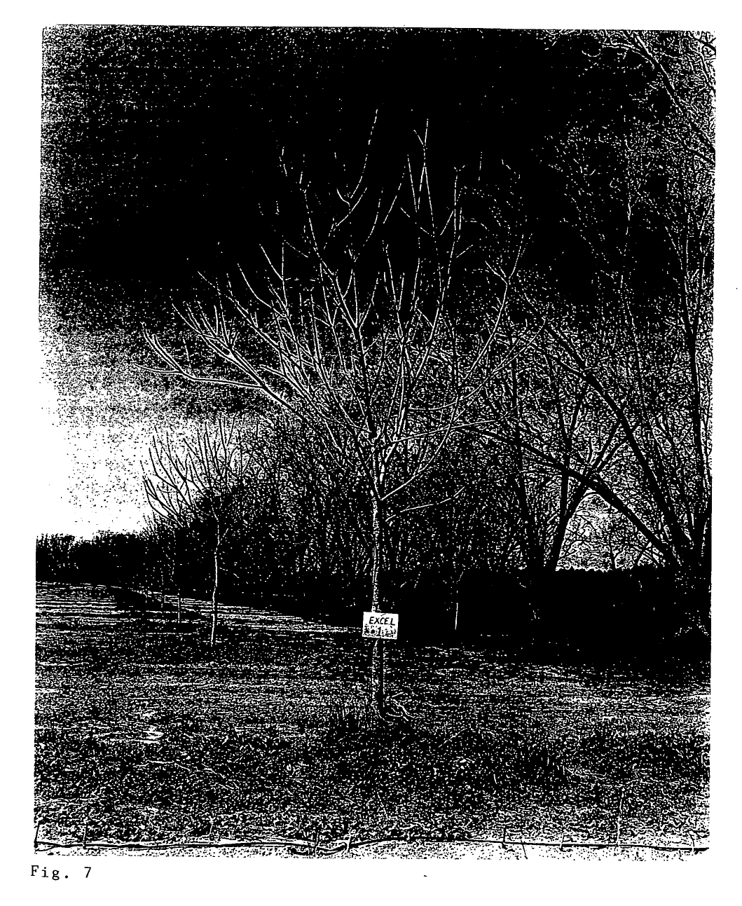

### DETAILED DESCRIPTION OF THE PREFERRED EMBODIMENTS

Having summarized various aspects of the present invention, reference will now be made in detail to the description of the invention as illustrated in the accompanying photographs.
The tree:
   Tree configuration.—Sparse, open tube growth showing small leaves and twig bits or semi dwarf and conducive to closer planting, displays a semi-dwarf type growth with small twiglets, foliage is somewhat sparse, which allows for better sunlight penetration and air circulation. Tree has good strong branch junctions.
   Foliage.—Foliage displays somewhat small leaflets which are of medium green color.
   Productivity.—Bearing age approximately 3 years, good regular bearer of quality nuts; trees have been shown to bear at 3 years after transplanting and shown increased heavy production, thereafter.
   Buds.—Buds are spaced somewhat far apart on twiglets, long & pointed.
   Catskins.—The final bloom is red and the catskins are long and thin Female bloom is red, catskins are somewhat light in color.
   Hardiness.—Tree blooms late and matures early leaving it less susceptible to frost damage.
   Resistance to disease and insects.—Nuts have shown high resistance to scab and other disease; foliage has shown heavy resistance to disease as well as good resistance to insects.
The nuts:
   Ripening.—Early bearing tree with fruit ready to shake about the 1^{st} week in October, which puts it about 2 weeks earlier that the standard Stuart variety. The tree actually blooms out later than Stuart but matures earlier. Schuck split begins around September 20th and nuts open uniformly in a matter of a few days.
   Maturity.—Nuts are ready to shake the 1st week in October.
   Quantity.—Nut count is about 45 per pound.
   Cluster size.—Generally 3 to 5.
   Configuration.—Nuts are in a tight cluster and shaped similar to a desirable variety.
   Shell.—Thick showing good bird resistance and adaptable to machine harvest.
   Kernel.—Excellent, bright meat, heavy oil content, yields about 51% with very consistent quality.
   Weight.—Some 7 year old trees have produced 50 pound or more.
   Size.—45 cmt. Nuts are larger and consistent with the general size and appearance of desirable variety.

DNA has been run on the “EXCEL” variety and it was shown to be both unique and dissimilar to any other known variety. Limbs grow in an upright position but to be willowy when heavily laden with nuts which tends to reduce excessive limb breakage. Because of the nut size and thickness of the shell the nut is bird resistant. This tree has been grafted and reproduced extensively on the farm of origin and the offspring has shown to be very consistent to the parent tree.

Because of its large size attractive shape, high quality meat kernel and early maturity, the new “EXCEL” variety should be an excellent choice for the in shell market as well as a good selection for commercial sellers.

1. A new and distinct variety of pecan tree substantially as shown and described herein, the new variety having a sturdy upright sparse tube showing small leaves and twig bits having a semi-dwarf type growth, the new variety being disease and insect resistant with excellent hardiness exhibiting a later bloom that the Stuart variety and an earlier maturing fruit, maturing about 14 days earlier than the Stuart variety, the fruit being of excellent kernel quality with bright meat and a heavy oil content.
